(19) United States
(12) Patent Application Publication
(10) Pub. No.: US 2015/0061832 A1
(43) Pub. Date: Mar. 5, 2015

(54) DIRECTLY OBSERVED THEARPY USING MOBILE TECHNOLOGY

(71) Applicant: UNIVERSITY OF UTAH RESEARCH FOUNDATION, Salt Lake City, UT (US)

(72) Inventors: Zeljko Pavlovic, Draper, UT (US); Catherine deVries, Salt Lake City, UT (US); Stacey Cole, St. Petersburg, FL (US)

(21) Appl. No.: 14/478,583

(22) Filed: Sep. 5, 2014

Related U.S. Application Data
(60) Provisional application No. 61/874,217, filed on Sep. 5, 2013.

(51) Int. Cl. G07C 9/00; G06K 9/00; A61B 5/117

(52) U.S. Cl. CPC G07C 9/00158; A61B 5/1172; G06K 9/00013
USPC 340/5.83

(57) ABSTRACT

The present disclosure provides systems and methods for performing directly observed therapy, and particularly, mobile directly observed therapy.

### BACKGROUND

Poor compliance in taking prescribed medications in the United States costs over $175 billion annually. Poor drug compliance in clinical trials can mean the difference between truly representative data and inaccurate, patient reported estimates. For infectious diseases, the risk to the community for poor compliance in infected individuals is significant. For this reason, in the 1990s, the World Health Organization adopted directly observed therapy, short-course (“DOTS”) as the recommended strategy for tuberculosis (“TB”) infection control. DOTS requires that a trained healthcare professional or other designated individual provides a prescribed drug and watches that patient take every dose. While DOTS has decreased the TB infection rate from 60% to 2% after treatment, a significant number of patients continue to suffer from TB that often persists due to medication non-compliance. In 2009 alone 5.8 million individuals throughout the world were diagnosed with TB. Non-compliance also contributes to drug-resistance strains of TB making this not just a population specific program, but a worldwide concern.

While DOTS has helped paved the way for improving medication compliance, it is still cumbersome in that it requires patients to actually ingest the drug in the presence of a healthcare provider, which is expensive, inconvenient and time intensive for both the patient and the provider. Moreover, patients that begin to improve may not have adequate incentive to continue with the program. Improved means for directly observing therapy may address these drawbacks.

### SUMMARY

The present disclosure provides methods and systems for performing mobile-directly observed therapy (“mDOT”), such as for clinical drug trials, law enforcement for medication compliance and treatment of disease (e.g., tuberculosis). mDOT provides a virtual salutation to performing directly observed therapy around the world.

Accordingly, the present disclosure provides systems for performing directly observed therapy, comprising a first computing device configured to: receive identification data over at least one network from a second computing device operated by a patient; receive verification of the identity of the patient based on the identification data; and upon receiving verification of the identity of the patient, transmit a signal to unlock at least one compartment of a medication dispensing device located with the patient. The identification data may include at least one of video data, audio data, a retinal scan, and a fingerprint scan. In some embodiments, the first computing device may be configured to store the received identification data. The first computing device also may be configured to transmit a second signal to the second computing device, the medication dispensing device, or both the second computing device and the medication dispensing device, with instructions to generate an alarm informing the patient to initiate a communication with the first computing device for transmitting the identification data.

For embodiments where the identification data includes video data, the first computing device may be configured to display the video data on a display device associated with the first computing device, and the first computing device may receive verification of the identity of the patient by receiving a manual verification input from a health representative reviewing the video data on the display device. The first computing device also may be configured to access a data file to retrieve a stored image of the patient, and to display the stored image on the display device so that the health representative can compare the video data to the stored image.

In some embodiments, the first computing device may be configured to receive verification of the identity of the patient by accessing a data file and retrieving stored identification data associated with the patient, comparing the received identification data to the stored identification data, and automatically verifying the identity of the patient when the received identification data substantially matches the stored identification data.

Some systems further may comprise the second computing device, the medication dispensing device, or both the second computing device and the medication dispensing device. The second computing device may include at least one of a smartphone, a tablet, a portable computer, and a desktop computer, and further may include at least one of a camera, a microphone, a retinal scanner and a fingerprint scanner. The second computing device may include an application configured to display a graphical user interface to the patient that enables the patient to initiate a communication with the first computing device for transmitting the identification data to the first computing device. In some embodiments, the second computing device and the medication dispensing device may be integrated into a single device.

The present disclosure also provides methods for performing directly observed therapy, comprising: receiving, by a first computing device, video data over at least one network from a second computing device operated by a patient; displaying, by the first computing device, the video data on at least one display device to at least one representative at a control center; receiving, by the first computing device, input from the representative of verification of the patient taking medication based on the video data; and upon receiving the input, issuing a reward to at least one account associated with the patient. In some embodiments, issuing a reward to at least one account includes adding minutes to a cellular telephone associated with the patient. In other embodiments, issuing a reward to at least one account includes adding funds to a monetary account associated with the patient.

### BRIEF DESCRIPTION OF THE DRAWINGS

FIG. 1 illustrates a system for performing directly observed therapy.

FIG. 2 is a drawing showing an exemplary patient operated device according to aspects of the present disclosure.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, the methods, operations, and sequences described herein can be performed in various orders. Therefore, unless otherwise indicated herein, no required order is to be implied from the order in which elements, steps, or limitations are presented in the detailed description or claims of the present application. Also unless otherwise indicated herein, the method and process steps described herein can be combined into fewer steps or separated into additional steps.

In addition, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

It should also be noted that a plurality of hardware and software based devices, as well as a plurality of different structural components may be used to implement the invention. In addition, it should be understood that embodiments of the invention may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (e.g., stored on non-transitory computer-readable medium) executable by one or more processors. As such, it should be noted that a plurality of hardware and software based devices, as well as a plurality of different structural components may be utilized to implement the invention. For example, “controllers” and “computing devices” described in the specification can include standard processing components, such as one or more processors, one or more non-transitory computer-readable medium modules, one or more input/output interfaces, and various connections (e.g., a system bus) connecting the components.

As noted above, embodiments of the invention provide systems and methods for performing mobile-directly observed therapy (“mDOT”), such as for clinical drug trials, law enforcement for medication compliance and treatment of disease (e.g., tuberculosis).

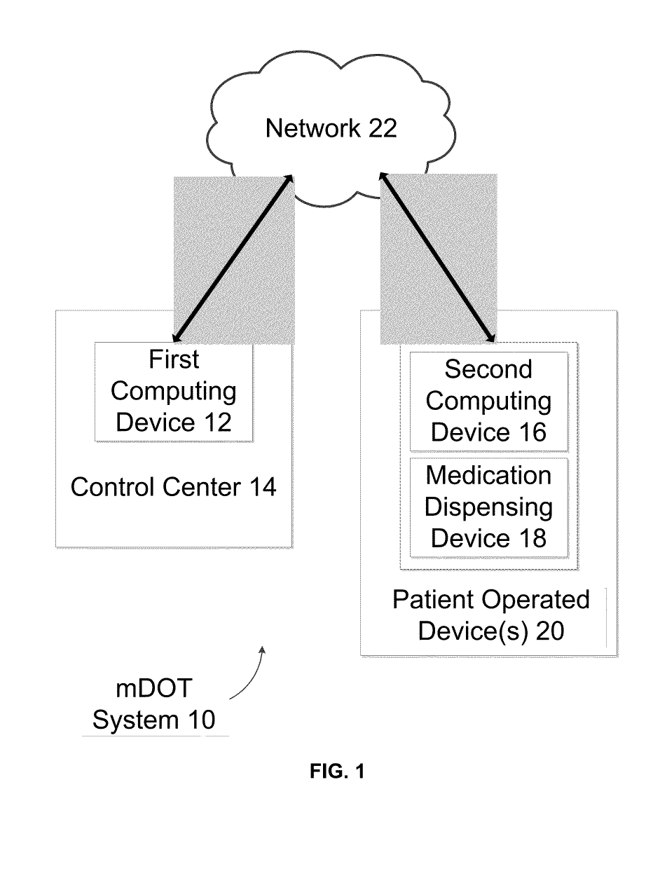

FIG. 1 illustrates an exemplary system 10 for performing mobile-directly observed therapy. The system may include one or more of a first computing device 12, a second computing device 16 and a medication dispensing device 18 operatively coupled to one another via at least one network 22. The first computing device 12 may be located at a control center 14, such as a call center, a healthcare facility, a pharmacy, or any other location where a healthcare provider could access a control system for implementing control software for controlling and implementing an mDOT program. The second computing device 16 may be located with and operated by a patient involved in an mDOT program, and may be used to communicate with the first communication device, as described in more detail below. The second computing device 16 may include a cell phone, a tablet computer, a portable computer, a desktop computer, or any other computing device configured to communicate with a remote device or system (e.g., using a wired and/or wireless connection) The medication dispensing device 18 also may be located with and operated by a patient. The second computing device 16 and the medication dispensing device 18 may be referred to as patient operated devices 20. In some cases, the second computing device and the medication dispensing device 18 may be integrated into a single computing device. The network 22 may include any wired and/or wireless network for communicating signals between devices.

In its most general sense, the mDOT system 10 may be used to remotely observe compliance with a treatment regimen. Specifically, the second computing device 16 may be used to transmit to the first computing device 12 video of the patient ingesting medication from the medication dispensing device. A healthcare provider can then remotely observe the video to verify that the patient has complied with the prescribed therapy. As described in more detail below, the mDOT system 10 may also include various mechanisms for ensuring and subsequently verifying that the proper patient ingests the proper medication at the proper time, thus complying with the therapy.

First, the mDOT system 10 may include mechanisms for verifying the identity of a patient, so that only one patient can access medication that is stored in a particular medication dispensing device 18. More specifically, the first computing device 12 may be configured to receive patient identification data over at least one network 22 from the second computing device 14; receive verification of the identity of the patient based on the identification data; and upon receiving verification of the identity of the patient, transmit a signal to unlock at least one compartment of a medication dispensing device 18 located with the patient. In the absence of proper identification of the patient, the first computing device 12 may not transmit a signal to unlock a compartment of the medication dispensing device 18, and as such, the medication dispensing device 18 may remain locked so as to inhibit access to the medication by anyone other than the patient.

Patient identification data may include at least one of video data, audio data, a retinal scan, and a fingerprint scan, and may be collected and transmitted to the first computing device 12 with a patient operated device 20. For example, the second computing device 16, the medication dispensing device 18, or an integrated device comprising both the second computing device 16 and the medication dispensing device 18, may include at least one of a camera for taking a picture or video of a patient, a microphone for recording a patient's voice, a retina scanner for scanning a patient's retina, a fingerprint scanner for scanning a patient's fingerprint, or any other device now known or hereinafter devised that could be used to collect identification data about a human patient. This identification data (along with associated metadata, such as timestamps, notes, patient identifiers, etc.) may then be transmitted to the first computing device, where the data may be captured and stored in a storage device (e.g., cloud based storage, or on a server at a call center) for later processing and/or for record keeping purposes.

After the first computing device 12 receives and/or stores the patient identification data, the system 10 may be used to verify the identity of the patient. For embodiments where the identification data includes video data (e.g., a photographic image or a movie file), the first computing device 12 may be configured to display the video data on a display device associated with the first computing device 12, where the video can be observed by a healthcare representative. The first computing device also may be configured to access a data file to retrieve a stored image of the patient, and to display the stored image on the display device so that the health representative can compare the video data to the stored image. After reviewing the video data, the healthcare representative may enter an input into the first computing device 12 indicating whether or not the identity of the patient has been verified. If the first computing device 12 receives this manual verification input, then the first computing device 12 may transmit a signal to the medication dispensing device 18 via the network 22 instructing the medication dispensing device 20 to unlock a compartment containing medication.

In some embodiments, software installed on the first computing device 12 may be used to assist healthcare representatives in verifying the identity of the patient. More specifically, the first computing device may be configured to receive verification of the identity of the patient by accessing a data file and retrieving stored identification data associated with the patient, comparing the received identification data to the stored identification data, and automatically verifying the identity of the patient when the received identification data substantially matches the stored identification data. For example, image processing software (e.g., facial recognition software) can be used to automatically compare video data received from someone alleging to be a particular patient to a stored image of that patient. Likewise, voice recognition software can be used to automatically compare an audio recording received form someone alleging to be a patient to a stored audio recording of that patient. Finally, fingerprint and retinal scan software can be used to compare fingerprint and retinal scans of those alleging to be a patient with stored fingerprints or retinal scans of that patient. If the data received at the first computing device 12 does not match with the stored data, then the system may be configured to alert the healthcare representative of a possible fraud situation. However, if the data received at the first computing device 12 matches the stored data, then the first computing device 12 may be configured to display a confirmation that prompts a healthcare professional to manually enter a verification input, upon which the first computing device 12 may transmit a signal to the medication dispensing device 18 via the network 22 instructing the medication dispensing device 20 to unlock a compartment containing medication. Alternatively, if the data received at the first computing device 12 matches the stored data, then the first computing device 12 may be configured to automatically transmit a signal to the medication dispensing device 18 via the network 22 instructing the medication dispensing device 20 to unlock a compartment containing medication.

After the patient has been verified and the medication dispensing device has been unlocked, the mDOT system 10 may be used to verify that the proper patient ingests the medication at the proper time, thus complying with the therapy. More specifically, the second computing device 16, the medication dispensing device 18 or an integrated device comprising both the second computing device 16 and the medication dispensing device 18 may include a camera used to take a video of the patient actually administering or ingesting the medication. This video may include additional metadata, including the date and time the video was taken, patient identification information, and the like. After the video has been taken, the patient operated device 20 used to take the video may be used to transmit the video via the network 22 to the first computing device 12, whereupon the video may be stored in a manner similar to the identification data (as described above). This video may be reviewed by a healthcare representative to ensure that the patient has complied with their therapy.

In some embodiments, image processing software may again be used to verify that the identity of the patient taking the medication. Image processing software also may be used to automatically determine the number and kinds of pills taken by the patient and/or whether the patient truly swallowed the pills. If the software identifies that the wrong kind or number of pills were (or are going to be) taken or that the patient did not actually swallow the pills, the software can alert the representative of a possible fraud situation. Also, in some embodiments, image processing software can be used on stored video data (e.g., to provide a redundant check or verification on medication adherence and/or for video conferencing that could not be handled in real-time or were uploaded to the cell center after being previously recorded).

The first computing device 12 also may be configured to transmit a second signal to the second computing device 16, the medication dispensing device 18, or both the second computing device 16 and the medication dispensing device 20, with instructions to generate an alarm informing the patient that it is time to take medication. This alarm may thereby prompt the patient to initiate a communication with the first computing device 12 for transmitting identification data, as described above. The alarm may be generated by the second computing device 16, the medication dispensing device 18, or both. Alternatively or additionally, the alarm may be generated by some other device, such as a device that is worn by a patient, including, but not limited to, a watch or a necklace. The times for the alarms may be programmed at a first computing device 12 operated at a control center 14, such as at a pharmacy or health care facility, such that the system 10 automatically transmits the second signal to the second computing device 16 at those times. A first computing device 12 also may be used to manually transmit a signal at a random time.

The second computing device 16 further may include an application configured to display a graphical user interface to the patient that may be used for communicating with the first computing device 12, and to enable the patient to control the patient operated device(s) 20. For example, the graphical user interface may be used to display automatic or manual messages received from the first computing device 12, to control acquisition and transmission of data with the patient operated device(s) 20, to prepare messages for sending to the control center 14, or for any other purpose consistent with the function of the patient operated devices. For example, a few days before the medication in the medication dispensing device 18 is scheduled to run out, the system 10 may automatically transmit a message to the patient notifying that they are near the end of their supply and will need to obtain a new supply of medication. In fact, the system 10 may be used to automatically or manually communicate any relevant information to the patient, including but not limited to, the time for medication refills or appointments, potential side-effects to be expected at certain times during the course of treatment, information about the patient's disease and the reason for close monitoring, and what to expect at the clinic, and the like. If the patient misses a dose, the system 10 may be configured to ask the patient (via text or video) why the dose was missed, and to forward the response (text or video) to the control center 14. The control center 14 can then contact the patient about the missed dose.

In the event that the mDOT system 10 malfunctions, an override code may be used by the patient to open the medication dispensing device 18 and access his or her medication (e.g., for one day). The override codes may be managed by the call center and provided to the patient by the call center in the case of malfunction.

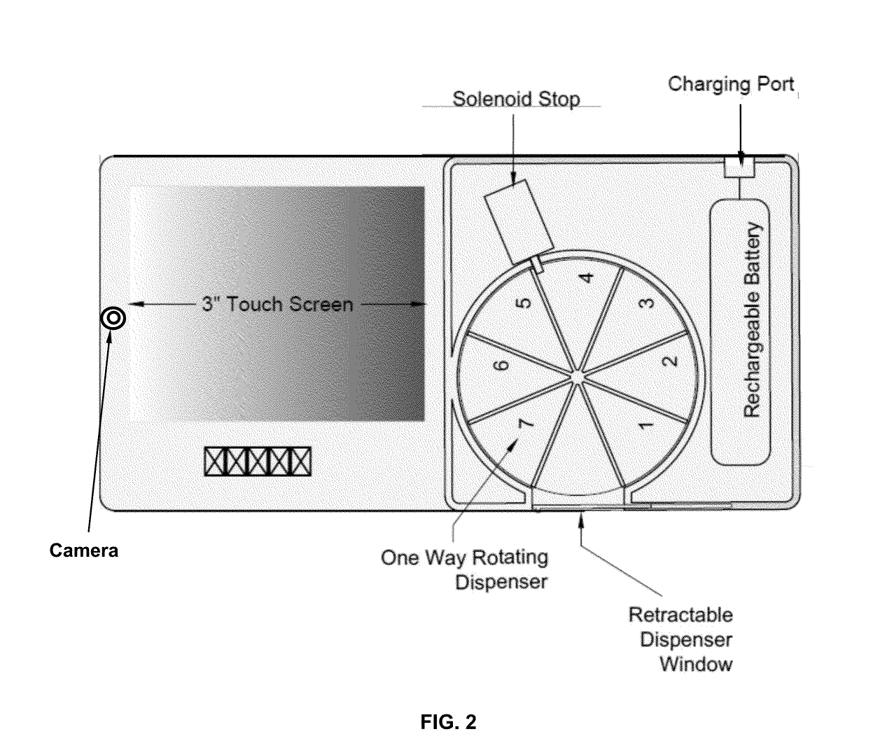

FIG. 2 shows an exemplary embodiment of a patient operated device 20, which includes a second computing device and a medication dispensing device integrated into a single unit. The device includes a camera that can be used to acquire video data (such as photographs or movies), a touch screen that can be used to control the device and communicate with a control center, and a medication dispenser (in the form of a one way rotating dispenser having a retractable dispenser window) that is controlled by a solenoid. The device may be used to acquire video data showing the identity of the patient, and to transmit that data to a first computing device at a control center for verification of the patient's identity, as described above. After the patient's identity has been verified, the device may receive a signal from the control center causing the solenoid to rotate the dispenser until a chamber (1-7) is positioned adjacent the retractable window. Medication within that chamber can then be removed by the patient. An application running on the device subsequently may cause the touch screen to display instructions for acquiring video of the administration or ingestion of the medication, and subsequently transmitting that video to the control center to verify that the patient has complied with their therapy.

The present disclosure also provides methods for performing directly observed therapy that includes a reward system. More specifically, the methods comprise: receiving, by a first computing device 12, video data over at least one network from a second computing device 16 operated by a patient; displaying, by the first computing device 12, the video data on at least one display device to at least one representative at a control center; receiving, by the first computing device 12, input from the representative of verification of the patient taking medication based on the video data; and upon receiving the input, issuing a reward to at least one account associated with the patient. In some embodiments, issuing a reward to at least one account includes adding minutes to a cellular telephone associated with the patient. In other embodiments, issuing a reward to at least one account includes adding funds to a monetary account associated with the patient (e.g., a bank account, an iTunes account, game credits, and the like). In some embodiments, the reward system is configured to issue a nominal reward for each successful mDOT medication verification and can further issue additional rewards at specific milestones for a certain level of compliance.

A sample procedure according to the present disclosure is described below. This procedure is merely exemplary, and is not intended to be limiting. Rather, numerous modifications may be made to this procedure according to other aspects of the disclosure described above.

At a predetermined time (e.g., approximately ten minutes) prior to scheduled medication time, an alarm sounds notifying the patient that it is nearly time for their medication. The patient uses a cell phone, tablet, laptop, or other device to open or launch an mDOT application stored on the device. The application generates a display (e.g., a graphical user interface) that includes a large button that the patient can select using a touchscreen included in the computing device or by selecting a certain tactile button on the device. The large button can indicate “call medication verification center.” Upon selecting the button, the application initiates a video call to the control center, and the patient is connected to the next available healthcare provider, who disables the alarm (if the alarm is not automatically disabled), and initiates a recording of the video data for storage. The identity of the patient is verified by comparing a picture of the patient with the person seen on the video (either with or without the use of specialized software to verify the patient's identity. Upon verification of the patient's identity, the healthcare worker enters an input indicating that the identity has been verified, upon which the system transmits a signal unlocking the patients medication dispensing device for that time and day. The healthcare worker then observes the patient remove the medication from the medication dispensing device after it is unlocked, and take the medication by placing it in his or her mouth and swallowing the medication. During the video interaction the health worker can also ask the patient if he/she has any additional concerns about the medication or has noticed any side-effects. If side-effects are noted, the health worker can either reassure the patient or refer the patient to a health professional if it appears the patient has had an adverse reaction. The health worker can also use the video feed to reinforce compliance, thanking the patient for taking their medication, and reminding them that a reward will be deposited into their virtual reward account within an indicated amount of time. In addition, if the patient has concerns or questions, information can be pushed to the system to provide education about the medication, side-effects or about their disease(s) in general. Prior to terminating the encounter, the individual medication compartment is relocked, and the video connection is terminated.

It should be understood that the systems and methods described herein can be used to directly observe any type of medication adherence. Furthermore, the systems and methods described here can be used to remotely observe and document any action of an individual or group of individuals that must adhere to a particular schedule or procedure.

Additional details can be found in the attached figures and the following claims.

We claim:

1. A system for performing directly observed therapy, the system comprising:
a first computing device configured to:
receive identification data over at least one network from a second computing device operated by a patient;
receive verification of the identity of the patient based on the identification data; and
upon receiving verification of the identity of the patient, transmit a signal to unlock at least one compartment of a medication dispensing device located with the patient.

2. The system of claim 1, wherein the first computing device is further configured to store the received identification data.

3. The system of claim 1, wherein the identification data includes at least one of video data, audio data, a retinal scan, and a fingerprint scan.

4. The system of claim 3, wherein the identification data includes video data, wherein the first computing device is configured to display the video data on a display device associated with the first computing device, and wherein the first computing device receives verification of the identity of the patient by receiving a manual verification input from a health representative reviewing the video data on the display device.

5. The system of claim 4, wherein the first computing device is further configured to access a data file to retrieve a stored image of the patient, and to display the stored image on the display device so that the health representative can compare the video data to the stored image.

6. The system of claim 3, wherein the first computing device is configured to receive verification of the identity of the patient by accessing a data file and retrieving stored identification data associated with the patient, comparing the received identification data to the stored identification data, and automatically verifying the identity of the patient when the received identification data substantially matches the stored identification data.

7. The system of claim 1, wherein the first computing device is configured to transmit a second signal to the second computing device, the medication dispensing device, or both the second computing device and the medication dispensing device, with instructions to generate an alarm informing the patient to initiate a communication with the first computing device for transmitting the identification data.

8. The system of claim 1, further comprising the second computing device, the medication dispensing device, or both the second computing device and the medication dispensing device.

9. The system of claim 8, wherein the second computing device includes at least one of a smartphone, a tablet, a portable computer, and a desktop computer, and further includes at least one of a camera, a microphone, a retinal scanner and a fingerprint scanner.

10. The system of claim 8, wherein the second computing device includes an application configured to display a graphical user interface to the patient that enables the patient to initiate a communication with the first computing device for transmitting the identification data to the first computing device.

11. The system of claim 8, wherein the second computing device and the medication dispensing device are integrated into a single device.

12. A method for performing directly observed therapy, the method comprising:
receiving, by a first computing device, video data over at least one network from a second computing device operated by a patient;
displaying, by the first computing device, the video data on at least one display device to at least one representative at a control center;
receiving, by the first computing device, input from the representative of verification of the patient taking medication based on the video data; and
upon receiving the input, issuing a reward to at least one account associated with the patient.

13. The method of claim 12, wherein issuing a reward to at least one account includes adding minutes to a cellular telephone associated with the patient.

14. The method of claim 12, wherein issuing a reward to at least one account includes adding funds to a monetary account associated with the patient.
